(19) United States
(12) Patent Application Publication
(10) Pub. No.: US 2005/0251338 A1
(43) Pub. Date: Nov. 10, 2005

(54) Method of soil geochemistry analysis prospecting

(75) Inventor: David Steven Thiede, St Lucia (AU)

Correspondence Address: BIRCH STEWART KOLASCH & BIRCH, PO BOX 747, FALLS CHURCH, VA, 22040-0747, US

(73) Assignee: SDP PTY LTD., CARSELDINE (AU)

(21) Appl. No.: 10/519,942

(22) PCT Filed: Jun. 30, 2003
(86) PCT No.: PCT/AU03/00832

§ 371 Date: Jul. 14, 2005

(30) Foreign Application Priority Data
Jul. 1, 2002 (AU) PS3297

(51) Int. Cl. G01N 33/24

(52) U.S. Cl. 702/2

(57) ABSTRACT

A method of soil-gas analysis prospecting including the steps of collecting a plurality of soil samples; subjecting each soil sample to soil-gas analysis for a plurality of signature gases to provide a signature gas value for each signature gas which together comprise a gas analysis subset for each sample; performing a multivariate discriminant analysis by providing for each sample a plurality of gas ratios by dividing a product of two or more signature gas values by a product of two or more signature gas value for each of the signature gases; and summing the gas ratios for each sample in the subset to provide a composite summed ratio parameter; and comparing the composite summed ratio parameter measured from the survey samples with the same parameter measured on samples having predetermined characteristics for a known mineralisation.

### FIELD OF INVENTION

THIS INVENTION relates to a method of soil geochemistry analysis prospecting. The present invention has particular application to soil-gas analysis using soil desorption pyrolysis, and for illustrative purposes, reference will be made to such application. It will be appreciated that the invention may have application to other soil geochemistry analysis techniques, particularly those which involve data sets with a large number of measured variables.

### BACKGROUND ART

Soil-gas analysis is an established, though not necessarily widely used, prospecting technique in which anomalies in the absorbed and/or adsorbed and/or pore-space gases in surface soils have been found to reflect mineralisation in the subsurface. However, previous techniques such as principal components analysis or cluster analysis have not been practical because the information characterising the mineralisation is not present in a high order variability. Additionally, sample preparation techniques have not addressed the inherent variability in analysis results caused by irrelevant components in the soil samples. Other soil geochemistry analysis techniques may have similar problems associated with multivariate analysis.

The present invention aims to provide a method of soil-gas analysis prospecting which addresses deficiencies in one or more of the sampling, the sample preparation techniques currently employed and/or the treatment of analysis data obtained from soil samples, or to provide a viable alternative method to present techniques for soil geochemistry analysis prospecting.

### DISCLOSURE OF THE INVENTION

With the foregoing in view, the present invention in one aspect resides broadly in a method of soil-gas analysis prospecting including the steps of:
collecting a plurality of soil samples;
subjecting each soil sample to soil-gas analysis for a plurality of signature gases to provide a signature gas value for each signature gas which together comprise a gas analysis subset for each sample;
providing for each sample a plurality of gas ratios by dividing a product of two or more signature gas values by a product of two or more signature gas value for each of the signature gases;
summing the gas ratios for each sample in the subset to provide a composite summed ratio parameter; and
comparing the composite summed ratio parameter measured from the survey samples with the same parameter measured on samples having predetermined characteristics for a known mineralisation.

Hereinafter, the providing of the gas ratios, and the summing of the gas ratios for each sample in the subset to provide a composite summed ratio parameter will be referred to as multivariate discriminant analysis as herein described.

In another aspect, the present invention resides broadly in a method of soil geochemistry analysis prospecting including the steps of:
collecting a plurality of soil samples;
separating selected component minerals from the samples to provide a corresponding plurality of component enriched samples;
subjecting each said component enriched sample to a geochemical analysis of a plurality of species discernable in said component enriched sample by said geochemical analysis, to provide a species analysis for each said component enriched sample and said species analyses together providing a composite analysis data set;
performing multivariate discriminant analysis as herein described on the composite analysis data set, and
comparing results of the multivariate discriminant analysis with one or more samples having a known mineralisation.

In another aspect, the present invention resides broadly in a method of soil geochemistry analysis prospecting including the steps of:
collecting a plurality of soil samples;
separating the clay minerals from the samples to provide a corresponding plurality of clay enriched samples;
subjecting each said clay enriched sample to an analysis of a plurality of adsorbed and/or absorbed species desorbable from said clay sample by said analysis, to provide a desorbed species analysis for each said sample and said desorbed species analyses together providing a composite analysis data set;
performing multivariate discriminant analysis as herein described on the composite analysis data set, and
comparing results of the multivariate discriminant analysis with one or more samples having a known mineralisation.

Preferably, the soil samples are treated to provide clay enriched samples which are subjected to a desorption process for desorbing desorbable species from the clay. It is further preferred that the desorption process includes soil desorption pyrolysis. In a preferred form, the gas ratios are provided by dividing a product of two gas values by a product of two other gas values. In such form, it is preferred that the soil or signature gas analysis is performed for forty-four signature gases using mass spectrometry. It will be appreciated that although the desorbed species may be referred to herein as “gas” or “gases”, other suitable states of desorbed species may be used in the method of the invention. Typically, the desorbed species to be analysed would be, for example, hydrocarbons or aliphatic sulfo-, sulfonyl or thionyl compounds or the like.

### BRIEF DESCRIPTION OF THE DRAWINGS

In order that the invention may be more readily understood and put into practical effect, reference will now be made to the following example which illustrates a preferred embodiment of the invention, and also to the accompanying drawings which illustrate the example and wherein:

FIG. 1 is a graph plotting the location of a number of soil samples to be used for soil-gas analysis prospecting according to the method of the invention;

FIG. 2 is a graph plotting the values obtained by desorption pyrolysis of a compound (“compound 04”) in respect of the samples of FIG. 1;

FIG. 3 is a graph plotting the values obtained by desorption pyrolysis of another compound (“compound 10”) in respect of the samples of FIG. 1;

FIG. 4 is a graph plotting the values obtained by desorption pyrolysis of a compound (“compound 19”) in respect of the samples of FIG. 1;

FIG. 5 is a graph plotting the values obtained by desorption pyrolysis of a compound (“compound 30”) in respect of the samples of FIG. 1;

FIG. 6 is a graph plotting the values obtained by dividing the product of the values of FIGS. 2 and 3 by the product of the values of FIGS. 4 and 5 in respect of the samples of FIG. 1;

FIG. 7 is a graph plotting the values obtained by subtracting a background value from the values of FIG. 6 in respect of the samples of FIG. 1; and

FIG. 8 is a graph plotting the values obtained by determining the relative sum of anomalous ratios in respect of the samples of FIG. 1.

### DETAILED DESCRIPTION OF THE DRAWINGS

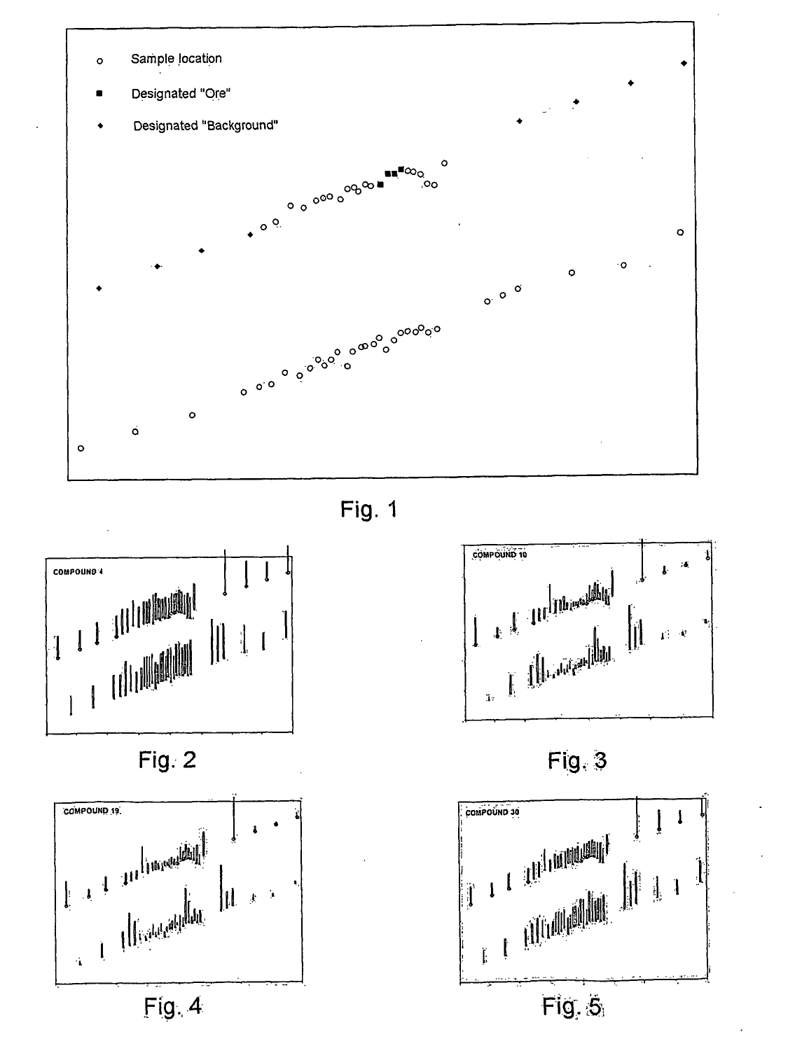

In the example, seventy-one soil samples were obtained from an area near Maronan, Queensland, along two lines represented by the markings shown in FIG. 1. Some of the samples were used as background and such samples have their respective plots marked “♦”, in order to provide a mineral defining function to discriminate between the inner group of samples from the outer group of samples. The samples represented by filled-in squares, viz. “▪” represent the samples having a known mineralisation. A clay enriched portion of each soil sample was separated and the clay enriched portions were subjected to pyrolysis desorption to a temperature of 450° C. and the desorbed material analysed for a number of compounds.

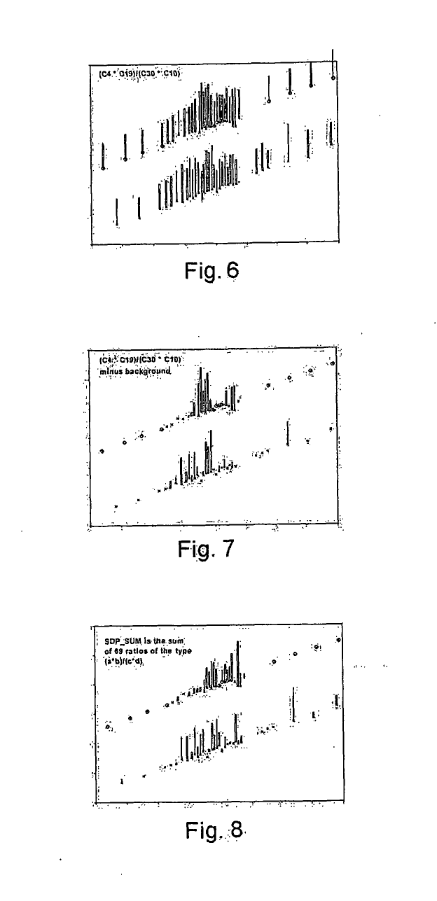

The values for each compound were processed in accordance with the method of the invention, one example of which is shown in FIGS. 2 to 7 in which four compounds were analysed for determined and treated by multiplying two pairs of values together and dividing their respective resulting products one into the other, and then subtracting a background value determined statistically from the individual values of the respective compounds to arrive at the plotted values shown in FIG. 7.

In the example, sixty-nine ratios of various compounds were determined in accordance with the invention and the results plotted in FIG. 8.

It can be seen in FIGS. 7 and 8 that some of the samples exhibit a values similar to the samples having a known mineralisation, particularly to the west of the area of known mineralisation as well as an area to the south of the area of known mineralisation.

The method of the present invention may be performed on soils in a wide variety of terrains in order to determine subsurface mineralisation without the need to drill many core samples to obtain more definitive mineralisation data. It will be appreciated that core samples would normally be obtained for area indicated by the method of the invention in order to confirm the mineralisation. However, the method of the present invention allows prospectors to be more selective in their core sample drillings, thereby lowering the cost of mineral exploration.

In use, the method of the present invention may be used to determine the mineralisation of a set of samples from soil geochemical analysis, particularly by employing multivariate discriminant analysis as herein described, taking appropriate care not to produce spurious mathematical artefacts.

Although the invention has been described with reference to a specific example, it will be appreciated by those skilled in the art that the invention may be embodied in other forms within the broad scope and ambit of the invention as claimed by the following claims.

1. A method of soil-gas analysis prospecting including the steps of:
collecting a plurality of soil samples;
subjecting each soil sample to soil-gas analysis for a plurality of signature gases to provide a signature gas value for each signature gas which together comprise a gas analysis subset for each sample;
performing a multivariate discriminant analysis by
providing for each sample a plurality of gas ratios by dividing a product of two or more signature gas values by a product of two or more signature gas value for each of the signature gases; and
summing the gas ratios for each sample in the subset to provide a composite summed ratio parameter; and
comparing the composite summed ratio parameter measured from the survey samples with the same parameter measured on samples having predetermined characteristics for a known mineralisation.

2. A method of soil geochemistry analysis prospecting including the steps of:
collecting a plurality of soil samples;
separating selecting component minerals from the samples to provide a corresponding plurality of component enriched samples;
subjecting each said component enriched sample to a geochemical analysis of a plurality of species discernable in said component enriched sample by said geochemical analysis, to provide a species analysis for each said component enriched sample and said species analyses together providing a composite analysis data set;
performing multivariate discriminant analysis according to claim 1 on the composite analysis data set, and
comparing results of the multivariate discriminant analysis with one or more samples having a known mineralisation.

3. A method of soil geochemistry analysis prospecting including the steps of:
collecting a plurality of soil samples;
separating the clay minerals from the samples to provide a corresponding plurality of clay enriched samples;
subjecting each said clay enriched sample to an analysis of a plurality of adsorbed and/or absorbed species desorbable from said clay sample by said analysis, to provide a desorbed species analysis for each said sample and said desorbed species analyses together providing a composite analysis data set;
performing multivariate discriminant analysis according to claim 1 on the composite analysis data set, and
comparing results of the multivariate discriminant analysis with one or more samples having a known mineralisation.

4. A method according to claim 1, wherein the soil samples are treated to provide clay enriched samples which are subjected to a description process for desorbing desorbable species from the clay.

5. A method according to claim 4, wherein the desorption process includes soil desorption pyrolysis.

6. A method according to claim 1, wherein the gas ratios are provided by dividing a product of two gas values by a product of two other gas values.

7. A method according to claim 6, wherein the soil or signature gas analysis is performed for forty-four signature gases using mass spectrometry.
